(19) United States
(12) Patent Application Publication
(10) Pub. No.: US 2011/0212086 A1
(43) Pub. Date: Sep. 1, 2011

(54) GITR Antibodies For The Treatment of Cancer

(75) Inventors: Srinivas Shankara, Shrewsbury, MA (US); Bruce Roberts, Southboro, MA (US)

(73) Assignee: GENZYME CORPORATION, Cambridge, MA (US)

(21) Appl. No.: 12/087,852

(22) PCT Filed: Jan. 19, 2007
(86) PCT No.: PCT/US07/60798

§ 371 Date: Jan. 20, 2009

Related U.S. Application Data
(60) Provisional application No. 60/761,010, filed on Jan. 19, 2006.

(51) Int. Cl. A61K 39/395; C12N 5/09; C12N 5/16; G01N 33/566; A61P 35/00

(52) U.S. Cl. 424/133.1; 435/375; 435/334; 435/7.23; 424/174.1; 424/144.1; 424/178.1

(57) ABSTRACT

The present invention provides compositions and methods for inhibiting the growth of a GITR-expressing cancer cell which cells may include, but are not limited to cells of epithelial origin such as NSCLC, prostate cancer, breast cancer, colon cancer and ovarian cancer and to treat or ameliorate the symptoms associated with the presence of these cells in a subject. Suitable compositions for use in these methods are antibodies that selectively recognize and bind to GITR (Glucocorticoid-induced TNFR-related protein) present on these cancer cells. The antibodies can be either polyclonal or monoclonal antibodies.

### TECHNICAL FIELD

The invention relates to compositions and methods for the treatment of human cancers and related malignancies.

### BACKGROUND

Despite numerous advances in medical research, cancer remains the second leading cause of death in the United States. In the industrialized nations, roughly one in five persons will die of cancer. Traditional modes of clinical care, such as surgical resection, radiotherapy and chemotherapy, have a significant failure rate, especially for solid tumors. Failure occurs either because the initial tumor is unresponsive, or because of recurrence due to regrowth at the original site and/or metastases.

Lung cancer is one of the most common malignancies worldwide and is the second leading cause of cancer death in man. See, American Cancer Society, Cancer facts and figures, 1996, Atlanta. Approximately 178,100 new cases of lung cancer were to be diagnosed in 1997, accounting for 13% of cancer diagnoses. An estimated 160,400 deaths due to lung cancer would occur in 1997 accounting for 29% of all cancer deaths, making lung cancer more deadly than the combination of breast, prostrate and colorectal cancers. Jemal, A. et al. (2004) Cancer Statistics 2004, CA: A Cancer Journal for Clinicians 53:5-26. The one-year survival rates for lung cancer have increased from 32% in 1973 to 41% in 1993, largely due to improvements in surgical techniques. The 5 year survival rate for all stages combined is only 14%. The survival rate is 48% for cases detected when the disease is still localized, but only 15% of lung cancers are discovered that early.

Among various forms of lung cancer, non-small cell lung cancer (NSCLC) accounts for nearly 80% of all new lung cancer cases each year. Small cell lung cancer is the most malignant and fastest growing form of lung cancer. The primary tumor is generally responsive to chemotherapy, but is followed by wide-spread metastasis. The median survival time at diagnosis is approximately 1 year, with a 5 year survival rate of 5%. For patients diagnosed with NSCLC, surgical resection offers the only chance of meaningful survival.

There are five types of non-small cell lung cancer: squamous cell carcinoma, adenocarcinoma, large cell carcinoma, adenosquamous carcinoma and undifferentiated carcinoma. Adenosquamous carcinomas begin in cells that appear flattened when viewed under a microscope. Undifferentiated carcinoma cells do not appear like normal cells and multiply uncontrollably. Squamous cell cancer is the most common type of lung cancer. It develops from the cells that line the airways. Adenocarcinoma develops from a glandular or secretory cells that produce mucus (phlegm). Large cell lung cancer has been thus named because the cells look large and rounded when they are viewed under a microscope.

Non-small cell cancer also is characterized by four clinical stages. Stage I is very localized cancer with no cancer in the lymph nodes. Stage II cancer has spread to the lymph nodes at the top of the affected lung. Stage III cancer has spread near to where the cancer started. This can be to the chest wall, the covering of the lung (pleura), the middle of the chest (mediastinum) or other lymph nodes. Stage IV cancer has spread to another part of the body.

Several antibody therapies are in development to treat lung cancer. Cetuximab and gifitinib are approved by the U.S. Food and Drug Administration for these cancers. Cetuximab in combination with chemotherapy has provided some benefit to NSCLC paticnts but further trials are still needed. Kelly, K. et al. (2003) Proc. Am. Soc. Clin. Oncol. 22:644.

Therefore, an effective treatment for NSCLC is still required. This invention satisfies this need and provides related advantages as well.

### DISCLOSURE OF THE INVENTION

The present invention provides compositions and methods for inhibiting the growth of a GITR-expressing cancer cell which cells may include, but are not limited to cells of epithelial origin such as NSCLC, prostate cancer, breast cancer, colon cancer and ovarian cancer and to treat or ameliorate the symptoms associated with the presence of these cells in a subject. Suitable compositions for use in these methods are antibodies that selectively recognize and bind to GITR (Glucocorticoid-induced TNFR-related protein) present on these cancer cells. The antibodies can be either polyclonal or monoclonal antibodies.

The antibodies of this invention are particularly useful to inhibit the growth of a cancer cell in vitro or in vivo, because Applicants have shown that antibodies useful in the methods of this invention recognize and bind GITR as well as possess at least one of the following characteristics:

promotion of complement-dependent cytotoxicity (CDC);

promotion of antibody-dependent cellular cytotoxicity (ADCC);

blocking of GITR ligand binding to GITR; or

promotion of down-regulation of T cell regulatory suppressor activity when administered to a subject in need thereof.

In a further aspect, the antibodies also promote apoptosis.

The invention also provides antibodies useful in the methods of this invention, wherein the antibodies recognize and bind GITR and promote down-regulation of T cell regulatory suppressor activity when administered to a subject in need thereof, and wherein the antibodies further possess at least one of the following characteristics:

promotion of complement-dependent cytotoxicity (CDC);

promotion of antibody-dependent cellular cytotoxicity (ADCC); or

blocking of GITR ligand binding to GITR.

In a further aspect, the antibodies also promote apoptosis.

Thus, in one aspect, the antibodies useful in the methods of this invention not only recognize and bind GITR, they also promote antibody-dependent cellular cytotoxicity (ADCC), e.g. antibodies A 06.

Thus, in one aspect, the antibodies useful in the methods of this invention not only recognize and bind GITR, they also block GITR-ligand binding and promote down-regulation of T cell regulatory suppressor activity when administered to a subject in need thereof, e.g., antibodies A 07.

Thus, in one aspect, the antibodies useful in the methods of this invention not only recognize and bind GITR, they also promote antibody-dependent cellular cytotoxicity (ADCC) and block GITR-ligand binding and promote down-regulation of T cell regulatory suppressor activity when administered to a subject in need thereof, e.g., antibodies A 10.

Thus, in one aspect, the antibodies useful in the methods of this invention not only recognize and bind GITR, they also promote complement-dependent cytotoxicity, e.g., antibodies A 11.

Thus, in one aspect, the antibodies useful in the methods of this invention not only recognize and bind GITR, they also promote complement-dependent cytotoxicity and promote antibody-dependent cellular cytotoxicity (ADCC) and block GITR-ligand binding, e.g., antibodies B 02 and B 03.

Thus, in one aspect, the antibodies useful in the methods of this invention not only recognize and bind GITR, they also promote complement-dependent cytotoxicity and block GITR-ligand binding and promote down-regulation of T cell regulatory suppressor activity when administered to a subject in need thereof, e.g., antibodies C 04.

Thus, in one aspect, the antibodies useful in the methods of this invention not only recognize and bind GITR, they also block GITR-ligand binding and promote down-regulation of T cell regulatory suppressor activity when administered to a subject in need thereof., e.g., antibodies D 02.

The functions of these antibodies are summarized in Table 1, below.

Biological Function(s) of Anti-GITR Antibodies

| | | | | Down |
|---|---|---|---|---|
| Internal | | | Inhibition of | Modulates |
| Antibody | | | GITR Ligand | Immune |
| Designation | CDC | ADCC | Binding | Tolerance |
| A 06 | | X | | |
| A 07 | | | X | X |
| A 10 | | X | X | X |
| A 11 | X | | | |
| B 02 | X | X | X | |
| B 03 | X | X | X | |
| C 04 | X | | X | X |
| D 02 | | | X | X |

The compositions useful in the methods of this invention can by polyclonal or monoclonal antibodies. The antibodies can be raised in any suitable species, e.g., murine, rat, bovine, simian or human. Variants, derivatives and functional fragments of these antibodies are also provided and can be used in the methods disclosed herein.

This invention also provides hybridoma cell lines that produce monoclonal antibodies having the above-noted specificities and biological function(s).

The antibodies and/or hybridoma cell lines can be combined with a carrier, for example, a pharmaceutically acceptable carrier for use in diagnostic and therapeutic methods.

This invention also provides methods to identify antibodies which can used in the methods of this invention with the same or similar to the function(s) identified in Table 1.

In one embodiment, the antibody of this invention is an IgG antibody and more particularly, the antibody is of a subclass of IgG selected from IgG₁, IgG₂, IgG₃ and IgG₄.

The invention also provides an antibody of the present invention conjugated to an antitumor agent or other therapeutic agent, or co-administrated with an antitumor agent, another therapeutic agent or radiation therapy. In some embodiments, the antitumor agent or therapeutic agent is a radionuclide.

The antibody can be an intact antibody molecule, scFv, a Fab fragment, or a F(ab′)₂ fragment. In some embodiments, the antibody is conjugated to an antitumor agent or other therapeutic agent, or co-administered with an antitumor agent, an antiangiogenic agent, or radiation therapy. In some embodiments, the agent is a radionuclide.

### BRIEF DESCRIPTION OF THE SEQUENCE LISTINGS

As used herein, the term “GITR gene” refers to at least the ORF of a contiguous polynucleotide sequence and that encodes a protein or polypeptide having the biological activity as set forth below. LocusLink, supra, reports that the protein encoded by this gene is a member of the TNF-receptor superfamily. This receptor has been reported to have increased expression upon T-cell activation, and it is thought to play a key role in dominant immunological self-tolerance maintained by CD25(+)CD4(+) regulatory T cells. Knockout studies in mice also suggest the role of this receptor is in the regulation of CD3-driven T-cell activation and programmed cell death. Three alternatively spliced transcript variants of this gene encoding distinct isoforms have been reported.

TABLE 2

| | Unigene & | Locus | Normal | | |
|---|---|---|---|---|---|
| | GenBank | Link | cell | Cancer cell | Seq. ID |
| Gene | Numbers | ID* | expression | Expression | Nos. |
| GITR | Hs.212680 | 8784 | | Adeno and | 1, 2 |
| (a/k/a. “TNFRSF18”) | AF117297.1 | | | squamous | |
| | AF241229.1 | | | cancers; | |
| | AF125304.1 | | | NSCLC; | |
| | AY358877.1 | | | ovarian, breast; | |
| | NM_148901.1 | | | prostate and | |
| | NM_148902.1 | | | colon cancers | |
| | NM_004195.2 | | | | |
| | NP_004186 | | | | |
| | NP_683699 | | | | |
| | NT_077913 | | | | |

*web address is = ncbi.nlm.nih.gov/LocusLink/list.cgi.

Sequence ID NO.: 1 is one example of a GITR polynucleotide, and others are known in the art, examples of which include, but are not limited to the sequences set forth in Table 2, and the sequences that encode GITR gene expression products as defined herein. Also included within this definition are biologically equivalent sequences such as those sequences that code for the polypeptide of SEQ ID NO:2 and those having at least 90% or alternatively, at least 95% sequence homology to an exemplary sequence, such as SEQ ID NO.: 1, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art. See for example, UniGene Cluster Hs.212680. Polynucleotide fragments are also known in the art, and include but are not limited to GenBank Accession numbers: BI911657.1; A1499936.1; A.1214481.1; and A1923712.1. These are particularly useful s as probes or primers.

As used herein, the term “GITR gene expression product, protein or polypeptide” includes the amino acid sequence of SEQ ID NO.: 2 as well as the amino acid sequences transcribed and translated from the GITR genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.:2 and which have the biological activity as described herein. Examples of homologous amino acid sequences include, but are not limited to polypeptides have the amino acid sequence of SEQ ID NO.: 2 or other GITR gene expression product that has been modified by conservative amino acid substitutions.

### MODES FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

Definitions

The practice of the present invention will employ, unless otherwise indicated, conventional techniques of immunology, molecular biology, microbiology, cell biology and recombinant DNA, which are within the skill of the art. See e.g., Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL and ANIMAL CELL CULTURE (R. I. Freshney, ed. (1987)).

As used herein, certain terms have the following defined meanings.

As used in the specification and claims, the singular form “a”, “an” and “the” include plural references unless the context clearly dictates otherwise. For example, the term “a cell” includes a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated. DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for guanine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

A “gene” refers to a polynucleotide containing at least one open reading frame (ORF) that is capable of encoding a particular polypeptide or protein after being transcribed and translated. Any of the polynucleotides sequences described herein may be used to identify larger fragments or full-length coding sequences of the gene with which they are associated. Methods of isolating larger fragment sequences are known to those of skill in the art.

A “gene product” or alternatively a “gene expression product” refers to the amino acid (e.g., peptide or polypeptide) generated when a gene is transcribed and translated.

The term “polypeptide” is used interchangeably with the term “protein” and in its broadest sense refers to a compound of two or more subunit amino acids, amino acid analogs or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D and L optical isomers, amino acid analogs and peptidomimetics. A peptide of three or more amino acids is commonly called an oligopeptide if the peptide chain is short. If the peptide chain is long, the peptide is commonly called a polypeptide or a protein.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” refers to a juxtaposition wherein the elements are in an arrangement allowing them to function.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention. Embodiments defined by each of these transition terms are within the scope of this invention.

The term “isolated” means separated from constituents, cellular and otherwise, in which the polynucleotide, peptide, polypeptide, protein, antibody or fragment(s) thereof, are normally associated with in nature. In one aspect of this invention, an isolated polynucleotide is separated from the 3′ and 5′ contiguous nucleotides with which it is normally associated with in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart. In addition, a “concentrated”, “separated” or “diluted” polynucleotide, peptide, polypeptide, protein, antibody or fragment(s) thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody or fragment(s) thereof, which differs from the naturally occurring counterpart in its primary sequence or for example, by its glycosylation pattern, need not be present in its isolated form since it is distinguishable from its naturally occurring counterpart by its primary sequence or, alternatively, by another characteristic such as glycosylation pattern. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known in the art to be capable of mediating transfer of genes to mammalian cells.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; recombinant yeast cells, metal particles; and bacteria or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts and may be used for gene therapy as well as for simple protein expression.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, “retroviral vector” refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof and a transgene. Adenoviruses (Ads) are a relatively well characterized, homogenous group of viruses, including over 50 serotypes. See e.g., WO 95/27071. Ads are easy to grow and do not require integration into the host cell genome. Recombinant Ad derived vectors, particularly those that reduce the potential for recombination and generation of wild-type virus, have also been constructed. See e.g., WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, recombinant yeast cells and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragment(s) thereof which bind cell surface antigens, e.g., TCR, CD3 or CD4.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes and proteins, including enzymes.

A “primer” is a short polynucleotide, generally with a free 3′ —OH group that binds to a target or “template” potentially present in a sample of interest by hybridizing with the target and, thereafter, promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer and a catalyst of polymerization, such as a DNA polymerase and, typically, a thermally-stable polymerase enzyme. Methods for PCR are well-known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra.

An expression “database” denotes a set of stored data that represent a collection of sequences, which in turn represent a collection of biological reference materials.

The term “cDNAs” refers to complementary DNA that is mRNA molecules present in a cell or organism made into cDNA with an enzyme such as reverse transcriptase. A “cDNA library” is a collection of all of the mRNA molecules present in a cell or organism, all turned into cDNA molecules with the enzyme reverse transcriptase, then inserted into “vectors” (other DNA molecules that can continue to replicate after addition of foreign DNA). Exemplary vectors for libraries include bacteriophage (also known as “phage”), viruses that infect bacteria, for example, lambda phage. The library can then be probed for the specific cDNA (and thus mRNA) of interest.

As used herein, “expression” refers to the process by which polynucleotides are transcribed into mRNA and/or the process by which the transcribed mRNA is subsequently being translated into peptides, polypeptides or proteins. If the polynucleotide is derived from genomic DNA, expression may include splicing of the mRNA in a eukaryotic cell. “Differentially expressed” as applied to a gene, refers to the differential production of the mRNA transcribed and/or translated from the gene or the protein product encoded by the gene. A differentially expressed gene may be overexpressed or underexpressed as compared to the expression level of a normal or control cell. However, as used herein overpression as at least 1.25 fold or, alternatively, at least 1.5 fold or, alternatively, at least 2 fold expression over that detected in a normal or healthy counterpart cell or tissue. The term “differentially expressed” also refers to nucleotide sequences in a cell or tissue which are expressed where silent in a control cell or not expressed where expressed in a control cell.

As used herein, “solid phase support” or “solid support”, used interchangeably, is not limited to a specific type of support. Rather a large number of supports are available and are known to one of ordinary skill in the art. Solid phase supports include silica gels, resins, derivatized plastic films, glass beads, cotton, plastic beads, alumina gels, microarrays and chips. As used herein, “solid support” also includes synthetic antigen-presenting matrices, cells and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel®, Rapp Polymere, Tubingen, Germany) or polydimethylacrylamide resin (obtained from Milligen/Biosearch, California).

A polynucleotide also can be attached to a solid support for use in high throughput screening assays. PCT WO 97/10365, for example, discloses the construction of high density oligonucleotide chips. See also, U.S. Pat. Nos. 5,405,783; 5,412,087; and 5,445,934. Using this method, the probes are synthesized on a derivatized glass surface also known as chip arrays. Photoprotected nucleoside phosphoramidites are coupled to the glass surface, selectively deprotected by photolysis through a photolithographic mask and reacted with a second protected nucleoside phosphoramidite. The coupling/deprotection process is repeated until the desired probe is complete.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding or in any other sequence-specific manner. The complex may comprise two strands forming a duplex structure, three or more strands forming a multi-stranded complex, a single self-hybridizing strand or any combination of these. A hybridization reaction may constitute a step in a more extensive process, such as the initiation of a PCR reaction or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

A polynucleotide or polynucleotide region (or a polypeptide or polypeptide region) has a certain percentage (for example, 80%, 85%, 90% or 95%) of “sequence identity” to another sequence means that, when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: www.ncbi.nlm.nih.gov/cgi-bin/BLAST.

Hyperplasia is a form of controlled cell proliferation involving an increase in cell number in a tissue or organ, without significant alteration in structure or function. Metaplasia is a form of controlled cell growth in which one type of fully differentiated cell substitutes for another type of differentiated cell. Metaplasia can occur in epithelial or connective tissue cells. Atypical metaplasia involves a somewhat disorderly metaplastic epithelium.

As used herein, the terms “neoplastic cells”, “neoplasia”, “tumor”, “tumor cells”, “cancer” and “cancer cells”, (used interchangeably) refer to cells which exhibit relatively autonomous growth, so that they exhibit an aberrant growth phenotype characterized by a significant loss of control of cell proliferation (i.e., de-regulated cell division). Neoplastic cells can be malignant or benign. A metastatic cell or tissue means that the cell can invade and destroy neighboring body structures.

As used herein the term “to inhibit” shall mean reduce or diminish the growth of a cell. In certain aspects the term is used synonymously with “suppressing growth of a cell.”

“Suppressing” cell growth indicates a growth state that is curtailed when compared to growth without therapeutic intervention. Tumor cell growth can be assessed by any means known in the art, including, but not limited to, measuring tumor size, determining whether tumor cells are proliferating using a ³H-thymidine incorporation assay or counting tumor cells. “Suppressing” cell growth means any or all of the following states: slowing, delaying and stopping tumor growth, as well as tumor shrinkage.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The term “antigen” is well understood in the art and includes substances which are immunogenic. The term as used herein also includes substances which induce immunological unresponsiveness or anergy.

A “native” or “natural” or “wild-type” antigen is a polypeptide, protein or a fragment which contains an epitope and which has been isolated from a natural biological source. It also can specifically bind to an antigen receptor.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein, any of which can be incorporated into an antibody of the present invention.

The antibodies can be polyclonal or monoclonal and can be isolated from any suitable biological source, e.g., murine, rat, sheep and canine. Additional sources are identified infra.

The term “antibody” is further intended to encompass digestion fragments, specified portions, derivatives and variants thereof, including antibody mimetics or comprising portions of antibodies that mimic the structure and/or function of an antibody or specified fragment or portion thereof, including single chain antibodies and fragments thereof. Examples of binding fragments encompassed within the term “antigen binding portion” of an antibody include a Fab fragment, a monovalent fragment consisting of the V_{L}, V_{H}, C_{L} and C_{H}, domains; a F(ab′)2 fragment, a bivalent fragment comprising two Fab fragments linked by a disulfide bridge at the hinge region; a Fd fragment consisting of the V_{H} and C_{H}, domains; a Fv fragment consisting of the V_{L} and V_{H} domains of a single arm of an antibody, a dAb fragment (Ward et al. (1989) Nature 341:544-546), which consists of a V_{H} domain; and an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv)). Bird et al. (1988) Science 242:423-426 and Huston et al. (1988) Proc. Natl. Acad Sci. USA 85:5879-5883. Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

As used herein, the term “antigen binding domain” refers to the part of an antibody molecule which comprises the area which specifically binds to and is complementary to part or all of an antigen. Where an antigen is large, an antibody may only bind to a particular part of the antigen, which part is termed an epitope. The antigen binding domain may be provided by one or more antibody variable domains (e.g., a Fd antibody fragment consisting of a V_{H} domain). Preferably, an antigen binding domain of an antibody comprises an antibody light chain variable region (V_{L}) and an antibody heavy chain variable region (V_{H}).

The term “antibody derivative” is intended to encompass molecules that bind an epitope as defined above and which are modifications or derivatives of a native monoclonal antibody of this invention. Derivatives include, but are not limited to, for example, bispecific, multispecific, heterospecific, trispecific, tetraspecific, multispecific antibodies, diabodies, chimeric, recombinant and humanized.

The term “bispecific molecule” is intended to include any agent, e.g., a protein, peptide, or protein or peptide complex, which has two different binding specificities. The term “multispecific molecule” or “heterospecific molecule” is intended to include any agent, e.g. a protein, peptide, or protein or peptide complex, which has more than two different binding specificities.

The term “heteroantibodies” refers to two or more antibodies, antibody binding fragments (e.g., Fab), derivatives thereof, or antigen binding regions linked together, at least two of which have different specificities.

The term “human antibody” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (V_{L}, V_{H})) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

The terms “monoclonal antibody” or “monoclonal antibody composition” as used herein refer to a preparation of antibody molecules of single molecular composition. A monoclonal antibody composition displays a single binding specificity and affinity for a particular epitope.

A “human monoclonal antibody” refers to antibodies displaying a single binding specificity which have variable and constant regions derived from human germline immunoglobulin sequences.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by heavy chain constant region genes.

The terms “transgenic, nonhuman animal” refers to a nonhuman animal having a genome comprising one or more human heavy and/or light chain transgenes or transchromosomes (either integrated or non-integrated into the animal's natural genomic DNA) and which is capable of expressing fully human antibodies. For example, a transgenic rat can have a human light chain transgene and either a human heavy chain transgene or human heavy chain transchromosome, such that the rat produces human anti-INF-α antibodies. The human heavy chain transgene can be integrated into the chromosomal DNA of the rat, or the human heavy chain transgene can be maintained extrachromosomally. Transgenic and transchromosomal animals are capable of producing multiple isotypes of human monoclonal antibodies to Alpha V (e.g., IgG, IgA and/or IgE) by undergoing V-D-J recombination and isotype switching.

A “composition” is also intended to encompass a combination of active agent and another carrier, e.g., compound or composition, inert (for example, a detectable agent or label) or active, such as an adjuvant, diluent, binder, stabilizer, buffers, salts, lipophilic solvents, preservative, adjuvant or the like. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term carrier further includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates cyclodextrins, such as 2-hydroxypropyl-.quadrature.-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional provisio that they be acceptable for use in vivo. For examples of carriers, stabilizers and adjuvants, see Martin REMINGTON'S PHARM. SCI., 15th Ed. (Mack Publ. Co., Easton (1975) and Williams & Williams, (1995), and in the “PHYSICIAN'S DESK REFERENCE”, 52^{nd} ed., Medical Economics, Montvale, N.J. (1998).

An “effective amount” is an amount sufficient to effect beneficial or desired results. An effective amount can be administered in one or more administrations, applications or dosages.

“Treating” or “treatment” or “alleviation” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for a polypeptide-expressing cancer if, after receiving a therapeutic amount of an anti-protein antibody, protein binding oligopeptide or protein binding organic molecule, the patient shows observable and/or measurable reduction in or absence of one or more of the following: reduction in the number of cancer cells or absence of the cancer cells; reduction in the tumor size; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues. To the extent the anti-protein antibody or protein binding oligopeptide may prevent growth and/or kill existing cancer cells, it may be cytostatic and/or cytotoxic. Reduction of these signs or symptoms may also be felt by the patient.

The above parameters for assessing successful treatment and improvement in the disease are readily measurable by routine procedures familiar to a physician. For cancer therapy, efficacy can be measured, for example, by assessing the time to disease progression (TTP) and/or determining the response rate (RR). Metastasis can be determined by staging tests and by bone scan and tests for calcium level and other enzymes to determine spread to the bone. CT scans can also be done to look for spread to the pelvis and lymph nodes in the area. Chest X-rays and measurement of liver enzyme levels by known methods are used to look for metastasis to the lungs and liver, respectively. Other routine methods for monitoring the disease include transrectal ultrasonography (TRUS) and transrectal needle biopsy (TRNB).

A “subject” is a vertebrate, preferably a mammal, more preferably a human. Mammals include, but are not limited to, murines, simians, humans, farm animals, sport animals, and pets.

A “control” is an alternative subject or sample used in an experiment for comparison purpose. A control can be “positive” or “negative”. For example, where the purpose of the experiment is to determine a correlation of an altered expression level of a gene with a particular type of cancer, it is generally preferable to use a positive control (a subject or a sample from a subject, carrying such alteration and exhibiting syndromes characteristic of that disease), and a negative control (a subject or a sample from a subject lacking the altered expression and clinical syndrome of that disease).

“Mammal” for purposes of the treatment of, alleviating the symptoms of or diagnosis of a cancer refers to any animal classified as a mammal, including humans, domestic and farm animals, and zoo, sports, or pet animals, such as dogs, cats, cattle, horses, sheep, pigs, goats, rabbits, etc. In one aspect, the term “mammal” is a human.

Administration “in combination with” one or more further therapeutic agents includes simultaneous (concurrent) and consecutive administration in any order.

Antibodies, Variants And Derivatives

The invention provides antibodies that recognize and specifically bind an epitope present on a GITR polypeptide or a variant as described above. The antibodies are distinguishable from known antibodies in that they possess different biological function(s) than those known in the art. For example, not only do the antibodies of the invention recognize and bind GITR, they are further characterized by having an additional biological activity selected from the ability to induce apoptosis is a cell that expresses or overexpresses GITR polypeptide, the ability to raise an antibody response (ADCC) or alternatively, the ability to raise a T cell response (CDC), or yet further, the ability to promote down-regulation of T cell regulatory suppressor activity or alternatively, to interfere with or block GITR-ligand binding. In further aspects, the antibodies are defined by possessing two or three of the above, but not all four. In a yet further aspect, the antibodies possess four biological activities.

As used herein, the term “selective binding” refers to the preferential binding of the antibody to GITR. Antibodies can be tested for selective and/or specific binding to GITR by comparing binding to commercially available GITR antibodies (e.g., BA689 (R&D Systems) to binding to irrelevant antigen or antigen mixture under a given set of conditions.

Also provided by this invention is an antibody, variant or derivative of the antibody which selectively binds an epitope present within the protein defined in Table 2 and having at least one additional biological function summarized in Table 1. In a further aspect, the antibodies promote apoptosis GITR expressing cells. The antibody can be a polyclonal or monoclonal antibody. Also provided are the hybridoma cell lines that produce the monoclonal antibodies of this invention.

The antibodies of this invention are monoclonal antibodies, although in certain aspects, polyclonal antibodies can be utilized. They also can be functional fragments, antibody derivatives or antibody variants. They can be chimeric, humanized, or totally human. A functional fragment of an antibody includes but is not limited to Fab, Fab′, Fab2, Fab′2, and single chain variable regions. Antibodies can be produced in cell culture, in phage, or in various animals, including but not limited to cows, rabbits, goats, mice, rats, hamsters, guinea pigs, sheep, dogs, cats, monkeys, chimpanzees, apes, etc. So long as the fragment or derivative retains specificity of binding or neutralization ability as the antibodies of this invention it can be used. Antibodies can be tested for specificity of binding by comparing binding to appropriate antigen to binding to irrelevant antigen or antigen mixture under a given set of conditions. Specific assays, e.g., ELISA, for determining specificity are known in the art. An example of an assay is provided in the Experimental Section, infra.

The monoclonal antibodies of the invention can be generated using conventional hybridoma techniques known in the art and well-described in the literature. For example, a hybridoma is produced by fusing a suitable immortal cell line (e.g., a myeloma cell line such as, but not limited to, Sp2/0, Sp2/0-AG14, NSO, NS1, NS2, AE-1, L.5, >243, P3X63Ag8.653, Sp2 SA3, Sp2 MAI, Sp2 SS1, Sp2 SA5, U397, MLA 144, ACT IV, MOLT4, DA-1, JURKAT, WEHI, K-562, COS, RAJI, NIH 3T3, HL-60, MLA 144, NAMAIWA, NEURO 2A, CHO, PerC.6, YB2/O) or the like, or heteromyelomas, fusion products thereof, or any cell or fusion cell derived therefrom, or any other suitable cell line as known in the art (see, e.g., www.atcc.org, www.lifetech.com., and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. Antibody producing cells can also be obtained from the peripheral blood or, preferably the spleen or lymph nodes, of humans or other suitable animals that have been immunized with the antigen of interest. Any other suitable host cell can also be used for expressing-heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from various commercial vendors such as Cambridge Antibody Technologies (Cambridgeshire, UK), MorphoSys (Martinsreid/Planegg, Del.), Biovation (Aberdeen, Scotland, UK) Biolnvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1977) Microbiol. Immunol. 41:901-907 (1997); Sandhu et al. (1996) Crit. Rev. Biotechnol. 16:95-118; Eren et al. (1998) Immunol. 93:154-161 that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al. (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass).; Gray et al. (1995) J. Imm. Meth. 182:155-163; Kenny et al. (1995) Bio/Technol. 13:787-790); B-cell selection (Steenbakkers et al. (1994) Molec. Biol. Reports 19:125-134 (1994).

Antibody variants of the present invention can also be prepared using delivering a polynucleotide encoding an antibody of this invention to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

The term “antibody variant” further includes post-translational modification to linear polypeptide sequence of the antibody or fragment. For example, U.S. Pat. No. 6,602,684 B1 describes a method for the generation of modified glycol-forms of antibodies, including whole antibody molecules, antibody fragments, or fusion proteins that include a region equivalent to the Fc region of an immunoglobulin, having enhanced Fc-mediated cellular toxicity, and glycoproteins so generated.

Antibody variants also can be prepared by delivering a polynucleotide of this invention to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al., Adv. Exp. Med. Biol. (1999) 464:127-147 and references cited therein. Antibody variants have also been produced in large amounts from transgenic plant seeds including antibody fragments, such as single chain antibodies (scFv's), including tobacco seeds and potato tubers. See, e.g., Conrad et al. (1998) Plant Mol. Biol. 38:101-109 and reference cited therein. Thus, antibodies of the present invention can also be produced using transgenic plants, according to know methods.

Antibody derivatives can be produced, for example, by adding exogenous sequences to modify immunogenicity or reduce, enhance or modify binding, affinity, on-rate, off-rate, avidity, specificity, half-life, or any other suitable characteristic. Generally part or all of the non-human or human CDR sequences are maintained while the non-human sequences of the variable and constant regions are replaced with human or other amino acids.

In general, the CDR residues are directly and most substantially involved in influencing antigen binding. Humanization or engineering of antibodies of the present invention can be performed using any known method, such as but not limited to those described in U.S. Pat. Nos. 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; and 4,816,567.

Techniques for making partially to fully human antibodies are known in the art and any such techniques can be used. According to one embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. (See for example, Russel, N. D. et al. (2000) Infection and Immunity April 2000:1820-1826; Gallo, M. L. et al. (2000) European J. of Immun. 30:534-540; Green, L. L. (1999) J. of Immun. Methods 231:11-23; Yang, X-D et al. (1999A) J. of Leukocyte Biology 66:401-410; Yang, X-D (1999B) Cancer Research 59(6):1236-1243; Jakobovits, A. (1998) Advanced Drug Delivery Reviews 31:33-42; Green, L. and Jakobovits, A. (1998) J. Exp. Med. 188(3):483-495; Jakobovits, A. (1998) Exp. Opin. Invest. Drugs 7(4):607-614; Tsuda, H. et al. (1997) Genomics 42:413-421; Sherman-Gold, R. (1997). Genetic Engineering News 17(14); Mendez, M. et al. (1997) Nature Genetics 15:146-156; Jakobovits, A. (1996) WEIR'S HANDBOOK OF EXPERIMENTAL IMMUNOLOGY, THE INTEGRATED IMMUNE SYSTEM VOL. IV, 194.1-194.7; Jakobovits, A. (1995) Current Opinion in Biotechnology 6:561-566; Mendez, M. et al. (1995) Genomics 26:294-307; Jakobovits, A. (1994) Current Biology 4(8):761-763; Arbones, M. et al. (1994) Immunity 1(4):247-260; Jakobovits, A. (1993) Nature 362(6417):255-258; Jakobovits, A. et al. (1993) Proc. Natl. Acad. Sci. USA 90(6):2551-2555; Kucherlapati, et al. U.S. Pat. No. 6,075,181.)

Human monoclonal antibodies can also be produced by a hybridoma which includes a B cell obtained from a transgenic nonhuman animal, e.g., a transgenic mouse, having a genome comprising a human heavy chain transgene and a light chain transgene fused to an immortalized cell.

The antibodies of this invention also can be modified to create chimeric antibodies. Chimeric antibodies are those in which the various domains of the antibodies' heavy and light chains are coded for by DNA from more than one species. See, e.g., U.S. Pat. No. 4,816,567.

The term “antibody derivative” also includes “diabodies” which are small antibody fragments with two antigen-binding sites, wherein fragments comprise a heavy chain variable domain (V_{H}) connected to a light chain variable domain (V_{L}) in the same polypeptide chain (V_{H} V_{L}). (See for example, EP 404,097; WO 93/11161; and Hollinger et al., (1993) Proc. Natl. Acad. Sci. USA 90:6444-6448.) By using a linker that is too short to allow pairing between the two domains on the same chain, the domains are forced to pair with the complementary domains of another chain and create two antigen-binding sites. (See also, U.S. Pat. No. 6,632,926 to Chen et al. which discloses antibody variants that have one or more amino acids inserted into a hypervariable region of the parent antibody and a binding affinity for a target antigen which is at least about two fold stronger than the binding affinity of the parent antibody for the antigen.)

The term “antibody derivative” further includes “linear antibodies”. The procedure for making the is known in the art and described in Zapata et al. (1995) Protein Eng. 8(10):1057-1062. Briefly, these antibodies comprise a pair of tandem Fd segments (V_{H}-C_{H}1-V_{H}-C_{H}1) which form a pair of antigen binding regions. Linear antibodies can be bispecific or monospecific.

The antibodies of this invention can be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

Antibodies of the present invention include naturally purified products, products of chemical synthetic procedures, and products produced by recombinant techniques from a eukaryotic host, including, for example, yeast, higher plant, insect and mammalian cells, or alternatively from a prokaryotic cells as described above.

In some aspects of this invention, it will be useful to detectably or therapeutically label the antibody. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample. Antibodies can also be conjugated, for example, to a pharmaceutical agent, such as chemotherapeutic drug or a toxin. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (¹³¹I), yttrium-90 (⁹⁰Y), bismuth-212 (²¹²Bi), bismuth-213 (²¹³Bi), technetium-99m (^{99m}Tc), rhenium-186 (186Re), and rhenium-188 (¹⁸⁸Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti cystic agents (e.g., antisense oligonucleotides, plasmids which encode for toxins, methotrexate, etc.); and other antibodies or antibody fragments, such as F(ab).

With respect to preparations containing antibodies covalently linked to organic molecules, they can be prepared using suitable methods, such as by reaction with one or more modifying agents. Examples of such include modifying and activating groups. A “modifying agent” as the term is used herein, refers to a suitable organic group (e.g., hydrophilic polymer, a fatty acid, a fatty acid ester) that comprises an activating group. Specific examples of these are provided supra. An “activating group” is a chemical moiety or functional group that can, under appropriate conditions, react with a second chemical group thereby forming a covalent bond between the modifying agent and the second chemical group. Examples of such are electrophilic groups such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., BIOCONJUGATE TECHNIQUES, Academic Press: San Diego, Calif. (1996)). An activating group can be bonded directly to the organic group (e.g., hydrophilic polymer, fatty acid, fatty acid ester), or through a linker moiety, for example a divalent C₁-C₁₂ group wherein one or more carbon atoms can be replaced by a heteroatom such as oxygen, nitrogen or sulfur. Suitable linker moieties include, for example, tetraethylene glycol. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl)carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate as described, or can be reacted with maleic anhydride and the resulting product cyclized to produce an activated maleimido derivative of the fatty acid.

The modified antibodies of the invention can be produced by reacting a human antibody or antigen-binding fragment with a modifying agent. For example, the organic moieties can be bonded to the antibody in a non-site specific manner by employing an amine-reactive modifying agent, for example, an NHS ester of PEG. Modified human antibodies or antigen-binding fragments can also be prepared by reducing disulfide bonds (e.g., intra-chain disulfide bonds) of an antibody or antigen-binding fragment. The reduced antibody or antigen-binding fragment can then be reacted with a thiol-reactive modifying agent to produce the modified antibody of the invention. Modified human antibodies and antigen-binding fragments comprising an organic moiety that is bonded to specific sites of an antibody of the present invention can be prepared using suitable methods, such as reverse proteolysis. See generally, Hermanson, G. T., BIOCONJUGATE TECHNIQUES, Academic Press: San Diego, Calif. (1996).

In one aspect, the antibodies are isolated. In another aspect, they are combined with a suitable carrier. The antibodies can be polyclonal or monoclonal and can be isolated from any species, including for example, murine, rat, simian, or recombinantly produced and isolated. Also provided by this invention are the hybridoma cell lines that produce these monoclonal antibodies, alone in combination with a carrier or in culture.

As is apparent to those of skill in the art, the antibodies can be sequenced and replicated by recombinant or synthetic means. They also can be further sequenced down to the linear sequence of nucleotides that encode them. Accordingly, this invention provides these polynucleotides, alone or in combination with a carrier, vector or host cell as described above, that encode a sequence of an antibody of this invention.

Also provided by this invention is a polypeptide that is attached to the terminal amine or carboxyl group of the peptide of a linear or branched sequence of an amino acid that has the biological activity or is derived from an antibody, variant, derivative or fragment thereof of the antibodies described above.

The antibodies of the present invention are useful in any suitable form that retains at least one desirable biologic activity of the intact antibody. In one embodiment, the antibody is Fab fragment, F(ab′)₂ fragment, a scFv, a diabody, a minibody, a nanobody, a multivalent single chain antibody, or an intact antibody molecule. “Single-chain Fv” or “scFv” antibody fragments comprise the V_{H} and V_{L} domains of the variable region of an antibody present in a single polypeptide chain. Generally, the scFv polypeptide further comprises a polypeptide linker between the V_{H} and V_{L} domains which enables the scFv to form the desired structure for antigen binding. See, e.g., Filipula et al., “Production of single chain Fv monomers or multimers” IN ANTIBODY ENGINEERING: A PRACTICAL APPROACH 253-68 (McCafferty et al., eds., Oxford University Press 1996). Fragments of intact molecules can be generated using methods well known in the art and include enzymatic digestion and recombinant means. See, e.g., CURRENT PROTOCOLS IN IMMUNOLOGY (Cooligan et al., eds., most recent edition). Fv, scFv or diabody molecules may be stabilized by the incorporation of disulphide bridges linking the V_{H} and V_{L} domains. See, e.g., Reiter et al. (1996) Nature Biotech. 14:1239-45. Diabodies comprising multivalent or multispecific fragments constructed by gene fusion may also be used with the antibodies disclosed herewith. See, e.g., U.S. Pat. No. 6,589,527; Holliger et al. (1993) Proc. Natl. Acad. Sci. USA 90:6444-48. Diabodies and scFv may be constructed without an Fc region, using only variable domains, potentially reducing the effects of anti-idiotypic reaction. Minibodies comprising a scFv joined to a CH₃ domain may also be made using the antibody of the present invention. See, e.g., U.S. Pat. No. 5,837,821 and Hu et al.(1996) Cancer Res. 56:3055-61. Nanobodies comprising single variable region (V_{H}) domain, originally characterized in camels and llamas can also be employed. See, e.g., Davies et al (1995) BioTechnology 13:475-79; Cortez-Retamozo, et al. (2004) Cancer Res. 64:2853-57.

The variable region and the associated CDR's of the present invention include completely identical sequences as well as those variants that retain the specificity of the source antibody. Thus, the variable region sequence and its associated CDR's may have one or more different amino acids from the source sequence. In one embodiment, the antibody is an IgG antibody, preferably an IgG₁ antibody. In a specific embodiment, the antibody is human, humanized, or chimeric. Alternatively, human anti-GITR antibodies can be murinized for greater cross-reactivity in murine disease models. Well known methods can be used to generate such antibodies. See, e.g., ANTIBODY ENGINEERING: A PRACTICAL APPROACH (McCafferty et al., eds., Oxford University Press 1996).

The identification and employment of a CDR or a set of CDR's of the invention will generally be that of an antibody heavy or light chain sequence or substantial portion thereof in which the CDR or set of CDR's is located at a location corresponding to the CDR or set of CDR's of naturally occurring V_{H} and V_{L} antibody variable domains encoded by rearranged immunoglobulin genes. The structures and locations of immunoglobulin variable domains may be determined by reference to Kabat et al., SEQUENCES OF PROTEINS OF IMMUNOLOGICAL INTEREST, US Department of Health and Human Services (4th Ed. 1987). Variable domains employed in the invention may be obtained from any germ-line or rearranged human variable domain, or may be a synthetic variable domain based on consensus sequences of known human variable domains. A CDR sequence of the invention may be introduced into a repertoire of variable domains lacking a CDR, using recombinant DNA technology.

For example, Marks et al. (1992) Bio/Technology 10:779-83 describe methods of producing repertoires of antibody variable domains in which consensus primers directed at or adjacent to the 5′ end of the variable domain area are used in conjunction with consensus primers to the third framework region of human V_{H} genes to provide a repertoire of VK variable domains lacking a CDR2. Marks et al. (1992) supra further describe how this repertoire may be combined with a CDR2 of a particular antibody. Using analogous techniques, the CDR3-derived sequences of the present invention may be shuffled with repertoires of V_{H} or V_{L} domains lacking a CDR3, and the shuffled complete V_{H} or V_{L} domains combined with a cognate V_{L} or V_{H} domain to provide specific binding members of the invention. The repertoire may then be displayed in a suitable host system such as the phage display system of, e.g., W092/01047; Kay et al. PHAGE DISPLAY OF PEPTIDES AND PROTEINS: A LABORATORY MANUAL (Academic Press 1996), so that suitable specific binding members may be selected. A repertoire may consist of from anything from 10⁴ individual members upwards, for example from 10⁶ to 10⁸ or 10¹⁰ members. Other suitable host systems include yeast display, bacterial display, T7 display, ribosome display and so on.

Additional antibody variants can be made by generating novel V_{H} or V_{L} regions carrying CDR-derived sequences of the invention using random mutagenesis of one or more selected V_{H} and/or V_{L} genes to generate mutations within the entire variable domain. One useful technique is error-prone PCR. See, e.g., Gram et al. (1992) Proc. Natl. Acad. Sci., USA 89:3576-80. In one embodiment, one or two amino acid substitutions are made within a set of heavy chain CDR's and/or light chain CDR's. Yet another method which may be used is to direct mutagenesis to CDR regions of V_{H} or V_{L} genes. Such techniques include, e.g., those disclosed in Barbas et al. (1994) Proc. Natl. Acad. Sci. USA 91:3809-13; Schier et al. (1996) J. Mol. Biol. 263:551-67.

Bispecific antibodies may also be used with the antibodies and their associated variable regions and CDR's as disclosed herein. These may be conventional bispecific antibodies and can be manufactured in a variety of ways that include, but is limited to being prepared chemically or from hybrid hybridomas. See, e.g., Holliger et al. (1993) Current Opinion Biotechnol. 4:446-49 (1993). Examples of bispecific antibodies include those of the BiTE™ technology in which the binding domains of two antibodies with different specificity can be used and directly linked via short flexible peptides. This combines two antibodies on a short single polypeptide chain. Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display from libraries. See, e.g., W094/13804. If one arm of the diabody is to be kept constant, for instance, with a specificity directed against a GITR polypeptide, then a library can be made where the other arm is varied and an antibody of appropriate specificity selected. In one embodiment, bispecific whole antibodies may be made by knobs-into-holes engineering. See, e.g., Ridgeway et al. (1996) Protein Eng. 9:616-21.

The antibodies comprising the variable region amino acid sequence or one or more CDR's of the GITR antibodies disclosed herein can be generated in cell culture, in phage, or in various animals, including but not limited to cows, rabbits, goats, mice, rats, hamsters, guinea pigs, sheep, dogs, cats, monkeys, chimpanzees, and apes. Therefore, the antibody useful in the present methods is a mammalian antibody. Phage techniques can be used to isolate an initial antibody or to generate variants with altered specificity or avidity characteristics. Such techniques are routine and well known in the art. In one embodiment, the antibody is produced by recombinant means known in the art. For example, a recombinant antibody can be produced by transfecting a host cell with a vector comprising a DNA sequence encoding the antibody. One or more vectors can be used to transfect the DNA sequence expressing at least one V_{L} and one V_{H} region in the host cell. Heavy and light chain variable region sequences can be joined to constant region sequence within an antibody expression vector. Such antibody expression vector is disclosed, for example, in U.S. Pat. No. 6,001,358. Heavy chain constant region of any class can be selected. The antibodies can be expressed in any suitable cell line. In one embodiment, the cell line is the CHO cell line. The preferred antibody class is IgG. The subclass of IgG can be selected from IgG₁, IgG₂, IgG₃ and IgG₄. The subclasses of IgG₁, IgG₂ and IgG₃ are preferred if ADCC and/or CDC, activity are necessary or expected for the desired therapeutic or biologic effect. When ADCC and/or CDC activity are necessary or expected for the desired therapeutic effect, IgG₁ is the most preferred embodiment.

A further aspect of the invention provides an antibody-antigen binding domain specific for GITR antigen, wherein the antibody-antigen binding domain is one of the domains of the disclosed antibodies with an addition, deletion, substitution or insertion of one or more amino acids in the amino acid sequence of a V_{H} domain of an antibody of the invention, wherein the V_{H} domain which is an amino acid sequence variant of the V_{H} domain that retains the desired functional activity of the original antibody. In some embodiments at least one amino acid of the heavy chain is deleted, added, or substituted with an amino acid different from the original amino acid. Any suitable method can be used to modify the antibodies of the present invention. Typically, the substitutions will be conservative substitutions. See, e.g., U.S. Pat. No. 5,624,821, U.S. Pat. No. 6,194,551, Application No. WO 9958572; and Angal, et al. (1993) Mol. Immunol. 30:105-08 (1993). The modified amino acid residues in the amino acid sequence of the heavy chain are 40% or less, preferably 30% or less, more preferably 20% or less within the entire heavy chain. Modifications can facilitate or induce enhancement of desirable biologic activities, e.g., increased. ADCC activity or serum half-life, or decrease an undesired biologic activity, e.g., immunogenicity. See, e.g., Sandlie et al., “Choosing and manipulating effector functions” IN ANTIBODY ENGINEERING: A PRACTICAL APPROACH 187-202 (McCafferty et al., eds., IRL Press 2002).

Epitope mapping can be performed to determine whether the antibody binds an epitope of interest. See, e.g., Champe et al. (1995) J. Biol. Chem. 270: 1388-94; EPITOPE MAPPING: A PRACTICAL APPROACH (Olwyn et al., Oxford University Press 2001). After hybridoma cells are identified that produce antibodies of the desired specificity, affinity, and/or activity, single-cell clones may be subcloned by limiting dilution procedures and grown by methods well known in the art. See, e.g., ANTIBODIES: A LABORATORY MANUAL (Harlow et al., eds., Cold Spring Harbor Laboratory 1988). Such antibodies can be human, humanized, or chimeric antibodies generated by well known techniques in the art.

The antibodies of the present invention can also be employed in heteroconjugate antibodies. As used herein, the term “heteroconjugate antibody” refers to two covalcntly joined antibodies. Such antibodies can be prepared using known methods in synthetic protein chemistry, including using crosslinking agents. See, e.g., U.S. Pat. No. 4,676,980.

It is also contemplated that any one or more antibody of the present invention may be conjugated to a bioactive agent. As used herein, the term “bioactive agent” refers to any synthetic or naturally occurring compound that enhances or mediates a desired biological effect. In one embodiment, the desired biological effect is stasis or cell death (e.g., apoptosis). In another embodiment, the desired biological effect results from the antibody sensitizing the target cell to a secondary agent that induces stasis or cell death.

One or more of the above can be further combined with a carrier, a pharmaceutically acceptable carrier or medical device which is suitable for use of the antibody or related composition in diagnostic or therapeutic methods.

The carrier can be a liquid phase carrier or solid phase carrier, e.g., bead, gel or carrier molecule such as a liposome. The composition can optionally further comprise at least one further compound, protein or composition.

An additional example of “carriers” includes therapeutically active agents such as another peptide or protein (e.g., an Fab' fragment). For example, an antibody of this invention, variant, derivative or fragment thereof can be functionally linked (e.g., by chemical coupling, genetic fusion, noncovalent association or otherwise) to one or more other molecular entities, such as another antibody (e.g., to produce a bispecific or a multispecific antibody), a cytotoxin, a cellular ligand or an antigen. Accordingly, this invention encompasses a large variety of antibody conjugates, bi- and multispecific molecules, and fusion proteins, whether or not they target the same epitope as the antibodies of this invention.

Yet additional examples of carriers are organic molecules (also termed modifying agents) or activating agents, that can be covalently attached, directly or indirectly, to an antibody of this invention. Attachment of the molecule can improve pharmacokinetic properties (e.g., increased in vivo serum half-life). Examples of organic molecules include, but are not limited to a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane.

Hydrophilic polymers suitable for modifying antibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. A suitable hydrophilic polymer that modifies the antibody of the invention has a molecular weight of about 800 to about 150,000 Daltons as a separate molecular entity. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying antibodies of the invention can be saturated or can contain one or more units of unsaturation. Examples of such include, but are not limited to n-dodecanoate, n-tetradecanoate, n-octadecanoate, n-eicosanoate, n-docosanoate, n-triacontanoate, n-tetracontanoate, cis-.DELTA.9-octadecanoate, all cis-.DELTA.5,8,11,14-eicosatetraenoate, octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

In yet another aspect, the present invention provides a transgenic nonhuman animal, such as a transgenic mouse (also referred to herein as a “HuMAb mouse”), which expresses a fully human monoclonal antibody that neutralizes at least one protein subtype similar to an antibody of this invention as defined above. In a particular embodiment, the transgenic nonhuman animal is a transgenic mouse having a genome comprising a human heavy chain transgene and a human light chain transgcnc encoding all or a portion of an anti-alpha V antibody of the invention. Preferably, the transgenic nonhuman animal, e.g., the transgenic mouse, is capable of producing multiple isotypes of human monoclonal antibodies to an epitope of interest by undergoing V-D-J recombination and isotype switching. Isotype switching may occur by, e.g., classical or non-classical isotype switching.

Accordingly, in another embodiment, the invention provides isolated cells derived or isolated from a transgenic nonhuman animal as described above, e.g., a transgenic mouse, which express human antibodies. The isolated B-cells can then be immortalized by fusion to an immortalized cell to provide a source (e.g., a hybridoma) of human antibodies. These hybridomas are also included within the scope of the invention.

The present invention further provides at least one antibody method or composition, for diagnosing or monitoring the expression of a cell expressing GITR protein or polypeptide which has been correlated by Applicants to cancer, e.g. a sarcoma such as NSCLC, in a cell, tissue, organ, animal or patient and/or, prior to, subsequent to, or during a related condition, as known in the art and/or as described herein. They are also used to prognose or monitor disease progression.

Also provided is a composition containing at least one antibody of this invention, variant, derivative or fragment thereof, suitable for administration in an effective amount to modulate or ameliorate symptoms associated with the presence of cells overexpressing GITR in a subject. The presence of these cells has been correlated by Applicants to the presence of pathological cells such as cancer. In one aspect, the antibodies, variants, derivatives or fragments thereof treat at least one GITR-related condition in a cell, tissue, organ, animal or patient and/or, prior to, subsequent to, or during a related condition, as known in the art and/or as described herein. The compositions include, for example, pharmaceutical and diagnostic compositions/kits, comprising a pharmaceutically acceptable carrier and at least one antibody of this invention, variant, derivative or fragment thereof. As noted above, the composition can further comprise additional antibodies or therapeutic agents which in combination, provide multiple therapies tailored to provide the maximum therapeutic benefit.

Alternatively, a composition of this invention can be co-administered with other therapeutic and cytotoxic agents, whether or not linked to them or administered in the same dosing. They can be coadministered simultaneously with such agents (e.g., in a single composition or separately) or can be administered before or after administration of such agents. Such agents can include corticosteroids, nonsteroidal immune suppressants, antimalarials, and nonsteroidal anti-inflammatory drugs. The compositions can be combined with alternative therapies such as administration of corticosteroids, nonsteroidal immune suppressants, antimalarials, and nonsteroidal anti-inflammatory drugs.

The methods of this invention can be practiced either in vitro or in vivo. When practiced in vitro, the methods require contacting the cells with (e.g., administering or delivering to the cells) one or more antibodies and/or related therapeutic compositions, derivatives etc. containing the antibodies as described above.

The antibodies and compositions can be delivered by any suitable means and with any suitable formulation. Accordingly, a formulation comprising an antibody of this invention is further provided herein. The formulation can further comprise one or more preservative or stabilizer such as phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4., 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1., 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, and 1.0%).

As noted above, the invention provides an article of manufacture, comprising packaging material and at least one vial comprising a solution of at least antibody, variant, derivative or fragment thereof of this invention with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36,40, 48, 54, 60, 66, 72 hours or greater. The invention further comprises an article of manufacture, comprising packaging material, a first vial comprising at least one lyophilized antibody of this invention and a second vial comprising an aqueous diluent of prescribed buffer or preservative, wherein said packaging material comprises a label that instructs a patient to reconstitute the antibody in the aqueous diluent to form a solution that can be held over a period of twenty-four hours or greater.

The range antibody includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

The formulations of the present invention can be prepared by a process which comprises mixing at least one antibody of this invention and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing of the antibody and preservative in an aqueous diluent is carried out using conventional dissolution and mixing procedures. For example, a measured amount of at least one antibody in buffered solution is combined with the desired preservative in a buffered solution in quantities sufficient to provide the antibody and preservative at the desired concentrations. Variations of this process would be recognized by one of ordinary skill in the art, e.g., the order the components are added, whether additional additives are used, the temperature and pH at which the formulation is prepared, are all factors that can be optimized for the concentration and means of administration used.

The compositions and formulations can be provided to patients as clear solutions or as dual vials comprising a vial of lyophilized antibody that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available. Recognized devices comprising these single vial systems include those pen-injector devices for delivery of a solution such as BD Pens, BD Autojectore, Humaject®′ NovoPen®, B-D®Pen, AutoPen®, and OptiPen®, GenotropinPen®, Genotronorm Pen®, Humatro Pen®, Reco-Pen®, Roferon Pen®, Biojector®, iject®, J-tip Needle-Free Injector®, Intraject®, Medi-Ject®, e.g., as made or developed by Becton Dickensen (Franklin Lakes, N.J. available at bectondickenson.com), Disetronic (Burgdorf, Switzerland, available at disetronic.com; Bioject, Portland, Oreg. (available at bioject.com); National Medical Products, Weston Medical (Peterborough, UK, available at weston-medical.com), Medi-Ject Corp (Minneapolis, Minn., available at mediject.com).

Also provided by this invention is an antibody, variant or derivative of the antibody which is useful in the manufacture of a medicament for administration to a subject, such as a human patient, to treat or diagnose conditions described herein.

Screening Assays

The present invention also provides a screen for identifying leads, drugs, therapeutic biologics and methods for reversing the neoplastic condition of the cells or selectively inhibiting growth or proliferation of the cells described above. In one aspect, the screen identifies antibodies, lead compounds or biological agents which are useful for the treatment of malignancy, hyperplasia or metaplasia characterized by differential expression of GITR.

Thus, to practice the method in vitro, suitable cell cultures or tissue cultures are first provided. The cell can be a cultured cell or a genetically modified cell which differentially expresses the gene of interest associated with a neoplastic cell. Alternatively, the cells can be from a tissue biopsy. The cells are cultured under conditions (temperature, growth or culture medium and gas (CO₂)) and for an appropriate amount of time to attain exponential proliferation without density dependent constraints. It also is desirable to maintain an additional separate cell culture; one which does not receive the agent being tested as a control.

As is apparent to one of skill in the art, the method can be modified for high throughput analysis and suitable cells may be cultured in microtiter plates and several agents may be assayed at the same time by noting genotypic changes, phenotypic changes and/or cell death.

When the agent is a composition other than a DNA or RNA nucleic acid molecule, the suitable conditions comprise directly adding the agent to the cell culture or adding the agent to culture medium for addition. As is apparent to those skilled in the art, an “effective” amount must be added which can be empirically determined.

The screen involves contacting the agent with a test cell characterized by differential expression of the gene of interest and then assaying the cell for the level of the gene of interest expression. In some aspects, it may be necessary to determine the level of the gene of interest expression prior to the assay. This provides a base line to compare expression after administration of the agent to the cell culture. In another embodiment, the test cell is a cultured cell from an established cell line that differentially expresses a gene of interest. An agent is a possible therapeutic agent if gene expression is returned (reduced or increased) to a level that is present in a cell in a normal or non-neoplastic state, or the cell selectively dies, or exhibits reduced rate of growth.

In yet another aspect, the test cell or tissue sample is isolated from the subject to be treated and one or more potential agents are screened to determine the optimal therapeutic and/or course of treatment for that individual patient.

For the purposes of this invention, an “agent” is intended to include, but not be limited to a biological or chemical compound such as a simple or complex organic or inorganic molecule, a peptide, a protein or an oligonucleotide. A vast array of compounds can be synthesized, for example oligomers, such as oligopeptides and oligonucleotides and synthetic organic compounds based on various core structures; these compounds are also included in the term “agent”. In addition, various natural sources can provide compounds for screening, such as plant or animal extracts and the like. It should be understood, although not always explicitly stated that the agent is used alone or in combination with another agent, having the same or different biological activity as the agents identified by the inventive screen. The agents and methods also are intended to be combined with other therapies.

As used herein, the term “reversing the neoplastic state of the cell” is intended to include apoptosis, necrosis or any other means of preventing cell division, reduced tumorigenicity, loss of pharmaceutical resistance, maturation, differentiation or reversion of the neoplastic phenotypes as described herein. As noted above, lung cells having differential expression of a gene of interest that results in the neoplastic state are suitably treated by this method. These cells can be identified by any method known in the art that allows for the identification of differential expression of the gene.

When the agent is a nucleic acid, it can be added to the cell cultures by methods known in the art, which includes, but is not limited to calcium phosphate precipitation, microinjection or electroporation. Alternatively or additionally, the nucleic acid can be incorporated into an expression or insertion vector for incorporation into the cells. Vectors that contain both a promoter and a cloning site into which a polynucleotide can be operatively linked are well known in the art and briefly described infra.

Polynucleotides are inserted into vector genomes using methods well known in the art. For example, insert and vector DNA can be contacted, under suitable conditions, with a restriction enzyme to create complementary ends on each molecule that can pair with each other and be joined together with a ligase. Alternatively, synthetic nucleic acid linkers can be ligated to the termini of restricted polynucleotide. These synthetic linkers contain nucleic acid sequences that correspond to a particular restriction site in the vector DNA. Additionally, an oligonucleotide containing a termination codon and an appropriate restriction site can be ligated for insertion into a vector containing, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; versatile multiple cloning sites; and T7 and SP6 RNA promoters for in vitro transcription of sense and antisense RNA. Other means are well-known and available in the art.

One can determine if the object of the method, i.e., reversal of the neoplastic state of the cell, has been achieved by a reduction of cell division, differentiation of the cell or assaying for a reduction in gene overexpression. Cellular differentiation can be monitored by histological methods or by monitoring for the presence or loss of certain cell surface markers, which may be associated with an undifferentiated phenotype, e.g., the expression of the gene of interest.

Kits containing the agents and instructions necessary to perform the screen and in vitro method as described herein also are claimed.

When the subject is an animal such as a rat or mouse, the method provides a convenient animal model system which can be used prior to clinical testing of the therapeutic agent or alternatively, for lead optimization. In this system, a candidate agent is a potential drug if gene expression is returned to a normal level or if symptoms associated or correlated to the presence of cells containing differential expression of a gene of interest are ameliorated, each as compared to untreated, animal having the pathological cells. It also can be useful to have a separate negative control group of cells or animals which are healthy and not treated, which provides a basis for comparison.

Therapeutic Methods

Therapeutic agents provided by this invention, include, but are not limited to the antibodies described above that specifically recognize and lyse cells expressing GITR. One can determine if a subject or patient will be beneficially treated by the use of agents by screening one or more of the agents against tumor cells isolated from the subject or patient using methods known in the art.

In one embodiment, the therapeutic agent is administered in an amount effective to treat cancer of epithelial origin, e.g., cancers of the lung, (NSCLC), prostate, ovarian, breast, colon, brain, kidney, pancreas, stomach, skin (melanoma). Therapeutics of the invention can also be used to prevent progression from a pre-neoplastic or non-malignant state into a neoplastic or a malignant state.

Various delivery systems are known and can be used to administer a therapeutic agent of the invention, e.g., encapsulation in liposomes, microparticles, microcapsules, expression by recombinant cells, receptor-mediated endocytosis (See, e.g., Wu and Wu (1987) J. Biol. Chem. 262:4429-4432), construction of a therapeutic nucleic acid as part of a retroviral or other vector, etc. Methods of delivery include but are not limited to intra-arterial, intra-muscular, intravenous, intranasal and oral routes. In a specific embodiment, it may be desirable to administer the pharmaceutical compositions of the invention locally to the area in need of treatment; this may be achieved by, for example, and not by way of limitation, local infusion during surgery, by injection or by means of a catheter.

The agents identified herein as effective for their intended purpose can be administered to subjects or individuals susceptible to or at risk of developing a disease correlated to the differential expression of GITR. When the agent is administered to a subject such as a mouse, a rat or a human patient, the agent can be added to a pharmaceutically acceptable carrier and systemically or topically administered to the subject. In one aspect, to determine patients that can be beneficially treated, a tumor sample is removed from the patient and the cells are assayed for the differential expression of the gene of interest. Therapeutic amounts can be empirically determined and will vary with the pathology being treated, the subject being treated and the efficacy and toxicity of the agent. When delivered to an animal, the method is useful to further confirm efficacy of the agent. As an example of an animal model, groups of nude mice (Balb/c NCR nu/nu female, Simonsen, Gilroy, Calif.) are each subcutaneously inoculated with about 10⁵ to about 10⁹ hyperproliferative, cancer or target cells as defined herein. When the tumor is established, the agent is administered, for example, by subcutaneous injection around the tumor. Tumor measurements to determine reduction of tumor size are made in two dimensions using venier calipers twice a week. Other animal models may also be employed as appropriate.

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated and the subject being treated. Single or multiple administrations can be carried out with the dose level and pattern being selected by the treating physician. Suitable dosage formulations and methods of administering the agents can be found below.

While it is possible for the agent to be administered alone, it is preferable to present it as a pharmaceutical formulation comprising at least one active ingredient, as defined above, together with one or more pharmaceutically acceptable carriers therefor and optionally other therapeutic agents. Each carrier must be “acceptable” in the sense of being compatible with the other ingredients of the formulation and not injurious to the patient.

Formulations include those suitable for oral, rectal, nasal, topical (including transdermal, buccal and sublingual), vaginal, parenteral (including subcutaneous, intramuscular, intravenous and intradermal) and pulmonary administration. The formulations may conveniently be presented in unit dosage form and may be prepared by any methods well known in the art of pharmacy. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both and then, if necessary, shaping the product.

Formulations suitable for parenteral administration include aqueous and non-aqueous isotonic sterile injection solutions which may contain anti-oxidants, buffers, bacteriostats and solutes which render the formulation isotonic with the blood of the intended recipient; and aqueous and non-aqueous sterile suspensions which may include suspending agents, thickening agents and liposomes or other microparticulate systems which are designed to target the compound to blood components or one or more organs. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily subdose, as herein above-recited, or an appropriate fraction thereof, of an agent.

The following examples are intended to illustrate, but not limit, Applicants' invention.

Experimental Methods

Experiment No. 1—Expression Analysis

Upon receipt of the samples, the tissues were minced with crossed scalpels. The minced tissues are treated with collagenase and elastase until single cell suspensions were obtained. The single cell suspensions were washed several times and the red blood cells were lysed. The white blood cells were removed by exposure of the cell suspension to antibodies to CD64, CD45 and CD14 linked to magnetic beads. The epithelial cells were isolated using an antibody to BerEP4 linked to a magnetic bead. The endothelial cells were isolated using an antibody to CD31 linked to a magnetic bead. RNA was prepared from the epithelial cell and endothelial cell samples immediately.

The quality of the RNA from the epithelial and endothelial cells was assessed overall and by expression of specific markers for the cell types. Cytokeratin 18, von Willebrand factor, EF1, P1H12, hevin and cytokeratin 8 were used as markers to determine whether the RNA collected represented a relatively pure cell population of epithelial cells or endothelial cells. After the quality assessment of the RNAs, samples were selected for SAGE analysis.

LongSAGE™ was performed on the RNA samples to a depth of approximately 50,000 tags for each library using the methods disclosed in Nature Biotechnology (2002) 20:508-512. An in-depth bioinformatics analysis was undertaken to characterize the SAGE data based upon increased expression of mRNAs across tumor types and in some aspects, as compared with normal cells.

GITR, glucocorticoid-induced TNFR-related protein, is known to be expressed by activated T cells and Treg cells. GITR is also known as activation-inducible TNFR family receptor (AITR) and tumor necrosis factor receptor superfamily, member 18 (TNFRSF-18) (Stephens, G. L. et al. (2004) J. Immunol. 173:5008-5020 and Nocentini, G. et al. (1997) P.N.A.S. 94:6216-6221). GITR ligand binds to GITR and triggers NF-kappaB activation through TRAF2. The GITR—GITR ligand interaction interrupts TCR-CD3 activation—induced apoptosis in T cells and may be involved in cell survival. The GITR ligand is also known as AITRL, GITRL, TL6 and hGIRTL. GITR is a 228 amino acid transmembrane protein that is suggested to be similar to 4-1BB and CD27. GITR protein has a 19 amino acid signal sequence, 134 amino acid extracellular region with three cysteine-rich motifs, a 23 amino acid transmembrane segment and a 52 amino acid cytoplasmic domain. The GITR ligand is expressed by endothelial cells (including HUVEC), B1 lymphocytes, mature and immature dendritic cells, and macrophages (Stephens, G. et al. (2004) supra.). GITR is involved in the interactions between T-lymphocytes and endothelial cells and in the regulation of T-cell receptor-mediated cell death. GITR mediates NF-kappaB activation via the TRAF2/NIK pathway. GITR binds to TNF receptor-associated factor-1 (TRAF1), TRAF2 and TRAF3 but not to TRAPS and TRAF6 (Nocentini, G. et al. (1997) supra.).

GITR is expressed on CD4+CD25+ T cells and after interaction with GITRL down-regulates T regulatory suppressor activity. Targeting GITR on tumor cells and depletion of CD4+CD25+ T cells could potentiate the efficacy of active tumor specific therapy (Kohm, A. P. et al. (2004) J. Immunol. 172:4686-4690; Shimizu, J. et al. (2002) Nature Immunol. 3:135-142 and Yamaguchi, T. and Sakaguchi, S. Seminal Cancer Biol. (Dec. 20, 2005)).

RT-PCR of bulk tissue RNA from 55 lung tumors and 18 normal lung tissues indicated ≧2-fold increased levels of GITR RNA in 76% of tumors compared with normal tissues. By RT-PCR GITR has very minimal expression in a variety of normal tissues including breast, prostate, brain, heart, kidney, liver, salivary gland, spleen stomach, thymus and uterus. RT-PCR of bulk tissues RNA from a variety of tumors and corresponding normal tissues indicated ≧2-fold increased levels of GITR RNA in 50% of ovarian cancers (n=40), 25% of melanomas (n=22), 50% of prostate cancers (n=24), 20% of colon cancers (n=26), and 66% of breast cancers (n=23).

Experiment No. 2—GITR Binding Assays

After generation the panel of antibodies are screened using cell based assays to identify those having the biological activities identified above using a conventional ELISA. CHO-K1 cells were engineered to overexpress GITR and used as a positive control for staining with 1 μg/ml of each antibody. H358 and Hut78 cells endogenously express GITR and were therefore used as positive controls. Anti-GITR antibody BA689 (purchased from R & D Systems, Inc.) was used as a positive control and human IgG as a negative control.

Experiment No. 3—GITR-Ligand Blocking Assay

Antibodies that bind GITR were selected for further analysis using a conventional competition assay. Briefly, anti-GITR antibodies were tested for their ability to compete with a known anti-GITR antibody BA689 (R&D Systems, Inc.). Campath was used as a negative control. Cells were incubated with antibodies in concentrations of 0, 1, 5 or 10 μg/ml of BA689 piro to incubation with 10 μg/ml test antibody. Ligand binding was detected by bio-anti-GITR antibody (BA6943, purchased from R&D Systems, Inc.) and anti-PE-anti-MolgG (#115-116-071, purchased from Jackson Labs).

Experiment No. 4—Modulation of T Regulatory (“T reg”) Suppression (CD4+/CD25+) Cell Activity

Normal donor PBMC (12/6/05prep) were used to isolate CD4+CD25+ and CD4+CD25− cells using the CD4+CD25+ regulatory T cell isolation kit purchased from Myltenyi Biotec. The CD4+CD25+ cells were expanded using the Dynal Human T reg Expander Dynabeads (20 μl/100 μl medium:AIMV+10% hu AB) in the presence of rIL-2 at 500 U/ml final. On day 8, CD4+CD25+ and CD4+CD25− cells were used in a mixed leukocyte reaction after the expander beads were removed by the use of a magnet. Cocultures were established using 5e4 responder CD4+CD25− T cells and 1e4 allogenic dendritic cells (RLS1185 day 7 DCs) as stimulators in a 96 well U-bottom plate. In vitro expanded CD4+CD25+ Treg cells were added in ratios of 1:1, 0.5:1 and 0.25:1 (CD25+:CD25− ratio). In addition 5 μl/ml final of several GITR antibody clones were added to respective wells. The wells were pulsed with 1 μCi/well of 3H-thymidine on day 5, and cultured for an additional 18 hrs before harvest.

The amount of T cell proliferation or lack of it was measured by 3[H] thymidine incorporation. The trend expected was that the highest the CD25+:CD25− ratio the lower the proliferation in the well. The addition of GITR antibody seems to either lower or enhance the suppressive capacity of T reg cells over the no antibody control. Clones C 04 and D 02 supressed Treg cell activity and Clones A 10 and A 07 mediated some enhancement in Treg cell suppressor activity.

Experiment No. 5—ADCC Activity

⁵¹Cr labeled target cells were arrayed at 5,000 cells per well with 1 μg/ml (200 ng/well) of anti-GITR and control antibody. Human PBMC were used as a source of effector cells at 200:1 and 100:1 effector:target ratio. Cells were cultured at 37° C. for 18 hrs. The cells were spun down and 25 μl of the reaction supernatant was measured for chromium release. The percent lysis was calculated by the formula

$$\frac{( Experimental Lysis \text{-} Spontaneous Release )}{( Total Lysis \text{-} Spontaneous Release )} \times 100$$

The results are summarized in Table 1, supra.

Experiment No. 6—CDC Activity

CHO cells engineered to express GITR or endogenously expressing lung cancer cells (H358 cells) were plated at 1×10e5 cells per well. Normal human serum complement (purchased from Quidel) was added to a final dilution of 10% (v/v) and dilutions of anti-GITR antibodies were also added to make a final volume of 200 μl per well. The assay was incubated for 3 hours at 37° C. 5% CO₂ after which time the plate was centrifuged and 100 μl of supernatant was transferred to a fresh plate. 100 μl of LDH detection substrate (purchased from Roche) was added and the plate was read at 490 nm.

The results are summarized in Table 1, supra.

Experiment No. 7—Apoptosis Assay

Apoptosis is measured using methods known in the art and described for example, in Bradbury, et al. (2000) J. Immunol. Methods 240:79-92 and paragraphs [0210] through [0226] of U.S. Patent Publication No. 2004/0253708A1.

It is to be understood that while the invention has been described in conjunction with the above embodiments, that the foregoing description and the following examples are intended to illustrate and not limit the scope of the invention. Other aspects, advantages and modifications within the scope of the invention will be apparent to those skilled in the art to which the invention pertains.

Experiment No. 8—In Vivo Efficacy

Antibodies are then further screened for in vivo efficacy using a syngeneic tumor model or human tumor xenograph model after plantation with endogenously expressing GITR cells or cells engineered to express GITR as described above. Such assays and models are known to those of skill in the art, for example, Tumor Models in Cancer Research, Teicher, B. A. ed. in the series Cancer Drug Discovery and Development, Humana Press, 2004 and Lev. A. et al. (2004) PNAS 101(24):9051-9056.

The above noted experimental methods are exemplary only and one of skill in the art can substitute one or more with other known methods. Thus, the antibodies of this invention are not limited by the test or assay. Additional biological assays are described in: Stanton, C. A. et al. (2004) Blood 103(2):601-606 and Malinda, K. M. et al. (1999) Exp. Cell Res. 250:168-173 (migration assay); paragraphs [0210] through [0226] of U.S. Patent Publication No. 2004/0253708A1 (apoptosis); paragraphs [0183] through [0194] of U.S. Patent Publication No. 2004/0258685A1 (inhibition, anti-proliferation, blocking and epitope binding assays); Stanton, C. A. et al. (2004) supra (proliferation and cytotoxicity assays); Manches, O. et al. (2003) Blood 101(3):949-954 (apoptosis, phagocytosis and ADCC assays); and paragraph [0066] of U.S. Patent Publication 2004/0228859A1 (CDC assay).

It is to be understood that while the invention has been described in conjunction with the above embodiments, that the foregoing description and the following examples are intended to illustrate and not limit the scope of the invention. Other aspects, advantages and modifications within the scope of the invention will be apparent to those skilled in the art to which the invention pertains.

What is claimed is:

1. A method for inhibiting the growth of a cancer cell that expresses GITR comprising contacting the cell with antibody that selectively recognizes and binds the GITR and is further characterized by at least one additional biological function selected from the group consisting of:
promotes complement-dependent cytotoxicity (CDC);
promotes antibody-dependent cellular cytotoxicity (ADCC);
blocks GITR ligand binding to GITR; and
promotes down-regulation of T cell regulatory suppressor activity when administered to a subject in need thereof.

2. The method of claim 1, wherein the cancer cell is selected from the group consisting of brain, breast, colon, kidney, ovary, pancreas, peritoneum, prostate, stomach, skin (melanoma) and lung.

3. The method of claim 1, wherein the cancer cell is a non-small cell lung cancer cell (NSCLC).

4. The method of claim 1, wherein the antibody is a polyclonal antibody or a monoclonal antibody.

5. The method of claim 1, wherein the antibody is a monoclonal antibody.

6. The method of claim 1, wherein the antibody is conjugated to a chemotherapeutic drug, a toxin, or a label.

7. The method of claim 5, wherein the antibody of is an IgG1 antibody.

8. The method of claim 1, wherein the antibody is a humanized antibody.

9. A hybridoma cell line that produces the monoclonal antibody of claim 5.

10. A method for inhibiting the growth of a cancer cell that expresses GITR comprising contacting the cell with antibody that selectively recognizes and binds the GITR and promotes down-regulation of T cell regulatory suppressor activity when administered to a subject in need thereof, and is further characterized by at least one additional biological function selected from the group consisting of:
promotes complement-dependent cytotoxicity (CDC);
promotes antibody-dependent cellular cytotoxicity (ADCC); and
blocks GITR ligand binding to GITR.

11. The method of claim 10, wherein the cancer cell is selected from the group consisting of brain, breast, colon, kidney, ovary, pancreas, peritoneum, prostate, stomach, skin (melanoma) and lung.

12. The method of claim 10, wherein the cancer cell is a non-small cell lung cancer cell (NSCLC).

13. The method of claim 10, wherein the antibody is a polyclonal antibody or a monoclonal antibody.

14. The method of claim 10, wherein the antibody is a monoclonal antibody.

15. The method of claim 10, wherein the antibody is conjugated to a chemotherapeutic drug, a toxin, or a label.

16. The method of claim 14, wherein the antibody of an IgG1 antibody.

17. The method of claim 10, wherein the antibody is a humanized antibody.

18. A hybridoma cell line that produces the monoclonal antibody of claim 14.

19. The method of claim 1 or 10, wherein the antibody further induces apoptosis in the cancer cell.

20. A method for selecting an anti-GITR antibody of interest, the method comprising
a) determining the level of at least one cytotoxic activity against a lung tumor cell selected from the group consisting of antibody-dependent cellular cytotoxicity and complement-dependent cytotoxicity of (i) a candidate anti-GITR antibody and (ii) a control antibody;
b) determining the level of down-regulation of a T regulatory cell suppressor activity of (i) a candidate anti-GITR antibody and (ii) a control antibody; and
c) selecting a anti-GITR antibody from steps (a) and (b) above comprising (i) at least one cytotoxic activity greater than that of the control antibody and (ii)a level of down-regulation of T regulatory cell suppressor activity greater than that of the control antibody.

21. The method of claim 20, wherein step (a) is performed before, during or after step (b).

22. The method of claim 20, wherein determining the level of cytotoxic activity in step (a) or down regulation in step (b) of the candidate anti-GITR antibody is performed before, during or after determining the level of cytotoxic activity in step (a) or down regulation in step (b) of the control antibody.

23. A method for treating a subject in need thereof, comprising administering to the subject an effective amount of an antibody that selectively recognizes and binds GITR and is further characterized by at least one additional biological function selected from the group consisting of:
promotes complement-dependent cytotoxicity (CDC);
promotes antibody-dependent cellular cytotoxicity (ADCC);
blocks GITR ligand binding to GITR; and
promotes down-regulation of T cell regulatory suppressor activity when administered to a subject in need thereof.

24. A method for treating a subject in need thereof, comprising administering to the subject an effective amount of an antibody that selectively recognizes and binds GITR and promotes down-regulation of T cell regulatory suppressor activity when administered to a subject in need thereof and is further characterized by at least one additional biological function selected from the group consisting of:
promotes complement-dependent cytotoxicity (CDC);
promotes antibody-dependent cellular cytotoxicity (ADCC);
blocks GITR ligand binding to GITR; and
promotes down-regulation of T cell regulatory suppressor activity when administered to a subject in need thereof.

25. The method of claim 23 or 24, wherein the antibody further induces apoptosis in the cancer cell.

26. The method of claim 23 or 24, wherein the subject in need thereof suffers from a cancer selected from the group consisting of brain, breast, colon, kidney, ovary, pancreas, peritoneum, prostate, stomach, skin (melanoma) and lung.

27. The method of claim 23 or 24, wherein the cancer cell is a non-small cell lung cancer cell (NSCLC).

28. The method of claim 23 or 24, wherein the antibody is a polyclonal antibody or a monoclonal antibody.

29. The method of claim 23 or 24, wherein the antibody is a monoclonal antibody.

30. The method of claim 23 or 24, wherein the antibody is conjugated to a chemotherapeutic drug, a toxin, or a label.

31. The method of claim 29, wherein the antibody of is an IgG 1 antibody.

32. The method of claim 23 or 24, wherein the antibody is a humanized antibody.

33. A hybridoma cell line that produces the monoclonal antibody of claim 29.


SEQUENCE LISTING (2 sequences)

SEQ ID NO: 1 - DNA, 705 bp, Unknown
  GITR
atggcacagcacggggcgatgggcgcgtttcgggccctgtgcggcctggcgctgctgtgcgcgctcagcctgggtcagcgccccaccgggggtcccgggtgcggccctgggcgcctcctgcttgggacgggaacggacgcgcgctgctgccgggttcacacgacgcgctgctgccgcgattacccgggcgaggagtgctgttccgagtgggactgcatgtgtgtccagcctgaattccactgcggagacccttgctgcacgacctgccggcaccacccttgtcccccaggccagggggtacagtcccaggggaaattcagttttggcttccagtgtatcgactgtgcctcggggaccttctccgggggccacgaaggccactgcaaaccttggacagactgcacccagttcgggtttctcactgtgttccctgggaacaagacccacaacgctgtgtgcgtcccagggtccccgccggcagagccgcttgggtggctgaccgtcgtcctcctggccgtggccgcctgcgtcctcctcctgacctcggcccagcttggactgcacatctggcagctgaggaagacccagctgctgctggaggtgccgccgtcgaccgaagacgccagaagctgccagttccccgaggaagagcggggcgagcgatcggcagaggagaaggggcggctgggagacctgtgggtgtga

SEQ ID NO: 2 - protein, 234 aa, Unknown
  GITR
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRKTQLLLEVPPSTEDARSCQFPEEERGERSAEEKGRLGDLWV